SEQUENCE LISTING (1 sequence)

SEQ ID NO: 1 - protein, 36 aa, Human immunodeficiency virus
YTSLIHSLIEESQNQQEKNEQELLELDKWASLWNWF

(19) United States
(12) Patent Application Publication
(10) Pub. No.: US 2005/0143568 A1
(43) Pub. Date: Jun. 30, 2005

(54) Peptide synthesis using filter decanting

(76) Inventor: Mark A. Schwindt, Boulder, CO (US)

Correspondence Address: Roche Colorado Corporation, 2075 North 55th Street, Boulder, CO, 80301, US

(21) Appl. No.: 11/021,820

(22) Filed: Dec. 23, 2004

Related U.S. Application Data
(60) Provisional application No. 60/533,691, filed on Dec. 31, 2003.

(51) Int. Cl. C07K 1/10

(52) U.S. Cl. 530/412

(57) ABSTRACT

The invention provides methods for synthesizing peptides, which include a step of filter decanting. Filter decanting involves removing supernatant from a mixture containing a precipitated peptide. A filter decanting apparatus can be used to remove the supernatant. The invention also provides methods, such as deprotection methods, that can be performed prior to the filter decanting method.

### PRIORITY CLAIM

The present non-provisional patent Application claims priority under 35 USC §119(e) from U.S. Provisional Patent Application having Ser. No. 60/533,691, filed on Dec. 31, 2003, and titled PEPTIDE SYNTHESIS USING FILTER DECANTING, wherein said provisional patent application is commonly owned by the owner of the present patent application and wherein the entire contents of said provisional patent application is incorporated herein by reference.

### FIELD OF THE INVENTION

The present invention relates to the synthesis of peptides and methods for the isolation of peptides during the synthetic process. The invention also relates to improvements for the large-scale synthesis of peptides.

### BACKGROUND

Many methods for peptide synthesis are described in the literature (for examples, see U.S. Pat. No. 6,015,881; Mergler et al. (1988) Tetrahedron Letters 29:4005-4008; Mergler et al. (1988) Tetrahedron Letters 29:4009-4012; Kamber et al. (eds), Peptides, Chemistry and Biology, ESCOM, Leiden (1992) 525-526; Riniker et al. (1993) Tetrahedron Letters 49:9307-9320; Lloyd-Williams et al. (1993) Tetrahedron Letters 49:11065-11133; and Andersson et al. (2000) Biopolymers 55:227-250. The various methods of synthesis are distinguished by the physical state of the phase in which the synthesis takes place, namely liquid phase or solid phase.

In some cases, liquid and solid phase peptide synthesis procedures have been scaled up in order to produce peptides on a pilot plant scale or on an industrial scale. Scaled-up, or “large-scale” peptide synthesis procedures are typically performed to produce peptides that have a pharmaceutical utility (Bray, B. L., Nature Rev., 2:587-593 (2003)). In order to meet the needs of global health care, pharmaceutically useful peptides may require yearly production levels in over kilogram amounts. Due to the technical difficulties and costs associated with the production of peptides, in general, there is a great need to optimize processes associated with production of pharmaceutically useful peptides on a large-scale basis in order to make their widespread use in global health care economically feasible.

A number of challenges are associated with the synthesis of peptides on a large-scale. Significant amounts of expensive reagents, including solvents such as dichloromethane (DCM) and coupling reagents, such as HOBt, are typically used during large-scale synthesis. Significant amounts are used not only because a particular step requires that the reagent be present in a certain excess, but also that these steps are repeated many times during the overall process. From an economic standpoint, it is desired to maximize the effectiveness of these reagents and to minimize the quantity used during the process.

Furthermore, due to the scale-up in reaction volume, methods for the synthesis of particular peptides on a small-scale basis are not always applicable for the synthesis of particular peptides on a large-scale basis.

The synthesis of a peptide typically requires a multitude of steps, many of which may be performed in one or more pieces of equipment and might be repeated either in succession, in parallel, or in an overlapping manner, during the entire synthetic process. When performing peptide synthesis on a large-scale, one or more small improvements in any particular step can lead to significant gains in the overall cost reduction, time reduction, and quality of the procedure. These improvements are greatly needed in the field of peptide synthesis, where it is critical to establish whether the synthesis of a particular therapeutically useful peptide is economically feasible.

In particular there is a need for improvements for the synthesis of therapeutically useful peptides that are relatively long, such as peptides that are longer than 15, 20, or 25 amino acid residues. It is understood that the amount of time and materials involved in synthesizing these long peptides is very considerable. In many cases, due to technical issues, synthesis of these peptides occurs using a combination of solid phase and solution phase steps, typically known as a hybrid approach. Therefore, there is a need for improvements in the synthesis of relatively long peptides, such as peptides made by a hybrid approach in large-scale, pilot plant scale, and small scale procedures.

### BRIEF DESCRIPTION OF THE DRAWINGS

FIG. 1 illustrates a reaction vessel having a filter decanting system of the present invention.

### SUMMARY OF THE INVENTION

The present invention provides filter decanting methods for improved recovery and improved isolation of peptides. In one aspect, the invention provides a method for isolating a peptide that includes the steps of providing a mixture comprising a supernatant and a peptide precipitate, and then filter decanting the supernatant to isolate the peptide precipitate. The peptide precipitate can be formed by contacting a peptide in solution with a precipitating agent while agitating the mixture.

The filter decanting method provides many advantages in the synthesis of peptides, such as improving peptide quality and reducing the time needed for peptide recovery and isolation steps. This, in turn, allows other steps in a peptide synthesis procedure to be optimized, such as deprotection reactions.

Therefore, another aspect of the invention provides a method for deprotecting a peptide that includes the steps of contacting a peptide comprising side chain protecting groups with a deprotection composition having an acidolytic agent in an amount greater than 90/100 parts to form a deprotected peptide, precipitating the deprotected peptide to form a mixture, and then filter decanting the supernatant to isolate the peptide precipitate.

### DETAILED DESCRIPTION

All publications and patents mentioned herein are hereby incorporated by reference. The publications and patents disclosed herein are provided solely for their disclosure.

The terminology used herein is not intended to limit the scope of the invention. Throughout the text, including the appended claims, the singular forms “a,” “an,” and “the” include plural reference unless the context clearly dictates otherwise. Thus, for example, a reference to “an amino acid residue” is a reference to one or more amino acid residues and includes equivalents thereof known to those skilled in the art. In this invention, certain terms are used frequently, the meanings of which are provided herein. Unless defined otherwise, terms used herein have the same meaning as commonly understood to one of ordinary skill in the art in this field of technology. Some terms may also be explained in greater detail later in the specification.

The present invention provides filter decanting methods that can be performed as steps during the synthesis of peptides and peptide intermediates. In some embodiments the filter decanting is performed following a precipitation step. The filter decanting methods as described herein can be performed at times during solution-phase, solid-phase, or hybrid synthesis procedures where it is desired to separate a particular component(s), for example, a reagent(s) involved in a coupling reaction and/or a deprotection reaction, from the peptide.

In some embodiments, the methods described herein are performed in a scaled-up peptide synthesis procedure. Scaled-up procedures are typically performed to provide an amount of peptide useful for distribution. For example the amount of peptide in a scaled-up procedure can be 500 g, or 1 kg per batch or more, and more typically tens of kg to hundreds of kg per batch or more. In scaled-up synthetic procedures such as large-scale synthesis one or more large reaction vessels can be used. These can accommodate quantities of reagents such as resins, solvents, amino acids, and chemicals for various steps in the synthesis process, in a size that allows for production of peptides in amounts, for example, in the range of 100-500 kilograms or more.

The methods described herein are particularly suitable for improving aspects of the peptide synthesis, particularly for scaled-up procedures. Improvements include the reduction in processing (synthesis) time, improvements in the yield of intermediates and final products, improvement in product purity, and reduction in amount of reagents, solvents, and starting materials needed.

The filter decanting methods described herein can be suitable for the synthesis of peptides having, for example, different amino acid sequences, different lengths, and different chemical modifications. The filter decanting method is particularly useful in methods wherein a full-length peptide is supplied to the filter decanting step. A “full-length peptide” refers to a peptide that is complete in regards to its amino acid content, that is, a peptide product that contains a desired number of amino acids. Full-length peptides include mature peptide products, wherein no additional chemical modifications are needed, or peptide products wherein certain chemical (non-amino) acid modifications can be made. For example, a full-length peptide can still have protecting groups, such as side chain protecting groups. A full-length peptide can be obtained after coupling of the ultimate amino acid to a growing peptide chain during a solution phase synthesis reaction, or after coupling two or more peptide intermediates. The full-length peptide product can then be subject to a deprotection reaction to remove the protecting groups. Generally, at this point, the full-length peptide is no longer attached to a solid phase support such as a polystyrene resin. Following peptide deprotection, the precipitation and filter decanting steps can be performed. If necessary, the peptide can be subject to one or more processing steps to remove unwanted chemical groups or to add desired chemical groups.

The methods described herein can be applied in the synthesis of any peptide, for example, any peptide chain of amino acid residues that are chemically bound together. The amino acid residues of the peptide synthesized can be naturally occurring amino acid residues, non-natural amino acid residues, or combinations thereof. Suitable peptides also include peptide intermediates which include compounds having an amino acid backbone and that are typically subject to one or more additional steps in a peptide synthesis scheme, for example, the coupling of another amino acid, the coupling of another peptide, the removal of one or more protecting groups, or any chemical modification of the peptide.

Peptides used in the methods of the invention can include common and rare naturally-occurring L-amino acids, non-natural amino acids, and similar residues that can be incorporated into a peptide.

The twenty common naturally-occurring amino acids residues are represented by the one-letter symbols as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine). Naturally-occurring rare amino acid include, for example, selenocysteine and pyrrolysine.

Non-natural amino acids can be organic compounds having a similar structure and reactivity to that of a naturally-occurring amino acid can include, for example, D-amino acids, beta amino acids, gamma amino acids; cyclic amino acid analogs, propargylglycine derivatives, 2-amino-4-cyanobutyric acid derivatives, Weinreb amides of α-amino acids, and amino alcohols.

The filter decanting methods of the invention can be used for the synthesis of peptides that can be made using a solid-phase approach, a solution phase approach, or a combination of approaches. Exemplary peptides include, but are not limited to oxytocin; vasopressin analogues such as felypressin, pitressin, lypressin, desmopressin, terlipressin; atosiban; adrenocorticotropic hormone (ACTH); insulin, glucagon; secretin; calcitonins such as human calcitonin, salmon calcitonin, eel calcitonin, dicarba-eel calcitonin (elcatonin); luteinizing hormone-releasing hormone (LH-RH) and analogues such as leuprolide, deslorelin, triptorelin, goserelin, buserelin; nafarelin, cetrorelix, ganirelix, parathyroid hormone (PTH); human coriticotropin-releasing factor, ovine coriticotropin-releasing factor; growth hormone releasing factor; somatostatin; lanreotide, octreotide; thyrotropin releasing hormone (TRH); thymosin α-1; thymopentin (TP-5); cyclosporin; integrilin; and angiotensin-converting enzyme inhibitors such as enalapril and lisinopril.

In some embodiments, the invention relates to methods for the synthesis of the enfuvirtide peptide (also known as T-20) and peptides having enfuvirtide activity. Enfuvirtide is a peptide which corresponds to amino acid residues 638 to 673 of the transmembrane protein gp41 from the HIV-1_{L}AI isolate and has a 36 amino acid sequence. The sequence of the enfuvirtide (SEQ ID NO 1) peptide is shown below:
SEQ ID NO 1: YTSLIHSLIEESQNQQEKNEQELLELDKWASLWNWF

Enfuvirtide is an anti-retroviral drug used for the treatment of HIV-1 infection. Enfuvirtide functions to block fusion of the HIV-1 viral particle with host cells by blocking the conformational changes required for membrane fusion. Peptides having this type of activity are herein referred to as having enfuvirtide activity.

Enfuvirtide synthesis typically utilizes both solid and liquid phase procedures to synthesize and combine groups of specific peptide fragments to yield the enfuvirtide product (Bray, B. L., Nature Rev., 2:587-593 (2003)). The present invention provides methods for the improved synthesis of both enfuvirtide peptide intermediates and full-length enfuvirtide products. The methods of the invention also include the synthesis of peptides having enfuvirtide activity and peptide intermediates used to prepare peptides having enfuvirtide activity. Peptides having enfuvirtide activity are described in U.S. Pat. Nos. 5,464,933 and 5,656,480, and PCT Publication No. WO 96/19495.

In some embodiments, the filter decanting methods can be performed before, during, or after a solution phase peptide synthesis step. For example, solution phase synthesis generally involves the coupling, in solution, of an amino acid to another amino acid, the coupling of peptide to amino acid, or the coupling of two peptides together. Typically, in solution-phase synthesis, the amino acids or peptides are not coupled to a resin, as they would be in a solid phase synthesis procedure. For example, a solution phase step can be performed to couple an amino acid to a growing peptide chain and then the formed peptide can be provided to a step wherein the peptide is subject to precipitation and then filter decanting, as described herein.

By way of example, the filter decanting steps can be incorporated into a solution phase synthesis as follows. A solution phase reaction is performed to couple one amino acid to another. A next amino acid is coupled to the growing peptide. The steps of coupling are repeated to obtain a desired peptide. The peptide is subject to a deprotection reaction to remove protecting groups. The deprotected peptide is then subject to the precipitation with a precipitating agent. The precipitated mixture is then subject to filter decanting wherein the supernatant is removed. After the filter decanting step the peptide may be in a desired form or can be subject to an additional reaction.

The filter decanting methods can also be performed following a solid phase peptide synthesis step. The solid phase synthesis step typically involves a peptide or an amino acid coupled to an insoluble support resin. In solid phase, an amino acid residue is coupled to the resin and additional residues are subsequently coupled to build the peptide chain. At some point during solid phase synthesis, the peptide that has been formed is cleaved from the resin. At this point the peptide is preferably separated from the resin. Next the peptide is contacted with a precipitating agent to form a mixture with the precipitated peptide. Filter decanting is then performed on the mixture.

To illustrate this aspect, the following solid phase procedure can be performed. Solid phase synthesis using Fmoc chemistry is performed to prepare a peptide coupled to a resin. After the full-length peptide is synthesized on the resin, it is cleaved from the resin using a cleavage reagent to generate a peptide in solution that is in a protected form. The peptide is then separated from the resin and the peptide is contacted with a precipitating agent to provide a mixture containing the precipitated peptide. The mixture is then subject to a filter decanting step.

Methods for the synthesis of peptides using a solid-phase approach are well known in the art. Accordingly, the invention contemplates using any solid phase synthetic approach for preparing a peptide which can then be used in the filter decanting method as described herein.

For example, the peptide that is provided to the filter decanting method of the invention can be synthesized by SSPS techniques using standard FMOC protocols. See, for example, Carpin et al. (1970), J. Am. Chem. Soc. 92(19):5748-5749; Carpin et al. (1972), J. Org. Chem. 37(22):3404-3409, “Fmoc Solid Phase Peptide Synthesis,” Weng C. Chan and Peter D. White Eds. (2000) Oxford University Press Oxford Eng. The solid phase syntheses of the peptide fragment intermediates of the invention can be carried out on an acid sensitive solid support, such as a “Wang” resin, which comprises a copolymer of styrene and divinylbenzene with 4-hydroxymethylphenyloxymethyl anchoring groups (Wang, S. S. 1973, J. Am. Chem. Soc.). Other suitable resins include 2-chlorotrityl chloride resin (Barlos et al. (1989) Tetrahedron Letters 30(30):3943-3946), and 4-hydroxymethyl-3-methoxyphenoxybutyric acid resin (Richter et al. (1994), Tetrahedron Letters 35(27):4705-4706). The Wang, 2-chlorotrityl chloride, and 4-hydroxymethyl-3-methoxyphenoxy butyric acid resins can be purchased from Calbiochem-Novabiochem Corp., San Diego, Calif.

In another example, the filter decanting can be performed during or after a full-length peptide has been formed by the coupling of peptide intermediate fragments. The combination of solid-phase synthesis to generate side-chain protected peptide intermediates with solution phase synthesis to couple the peptide intermediates is typically known as a hybrid approach. The hybrid approach allows for the preparation of peptides that are often difficult to synthesize using a solid-phase approach or a solution-phase approach alone. The precipitation and filter decanting step of the invention would typically be performed in the final stages of a hybrid approach.

For example, peptide intermediate fragments are coupled together in solution to form a full-length peptide. The full-length peptide is subject to a deprotection reaction to remove protecting groups. The deprotected peptide is then precipitated and the precipitated mixture is filter decanted. After filter decanting, the peptide may be in a desired form or can be subject to an additional reaction. In a preferred embodiment, the precipitation and filter decanting methods described herein can be performed after the coupling of peptide intermediate fragments to make an enfuvirtide or enfuvirtide-like peptide.

The methods described herein provide procedures for the improved purification of a peptide from components utilized in peptide synthesis. The methods of the invention include steps of providing a precipitated peptide, or precipitating a peptide, wherein the precipitated peptide is in a mixture, and then subjecting the mixture to a filter decanting process.

In some embodiments, the method includes steps of contacting a peptide in solution with a precipitating agent to form a mixture that has a supernatant and a peptide precipitate, agitating the mixture, and then filter decanting the supernatant to isolate the peptide precipitate. The peptide can be supplied in solution in any suitable form to be precipitated, for example, the peptide can be a deprotected peptide in a solution that includes reacted deprotection reagents.

In embodiments wherein the peptide is precipitated, a precipitating agent, for example a nonsolvent or a precipitating compound that is in solution, is placed in contact the with the peptide. In exemplary procedures, both the precipitating agent and the peptide are in a liquid phase and the precipitating agent is mixed with the peptide.

In other embodiments, the peptide is provided to the filter decanting step in a precipitated form.

The step of filter decanting involves separating a precipitated peptide, which is in a mixture, from other components in the mixture. The other components can be any reagent that is used in one or more steps that precede the filter decanting, such as coupling reagents, deprotection reagents, or precipitating reagents. In the case where a precipitating step is performed, the precipitating agent can be added to the peptide, which is typically in a solution. Filter decanting is performed on the premise that the precipitated peptide particles can be separated from the mixture using filter decanting apparatus.

Filter decanting allows the precipitated peptide to be separated from the other components in the mixture very rapidly. This rapid separation protects sensitive peptides from potentially destructive reagents. Also, according to the methods described herein, the precipitated peptide can also be kept cold during the filter decant process. This is a significant advantage for the isolation and recovery of a peptide precipitate. During the filter decant process the peptide can be kept at a temperature in the range of, for example, between −15° C. to +5° C. This significantly improves the quality of the peptide by minimizing its degradation. The actual temperature, or temperature range, that the filter decant process is performed at can vary depending on the sensitivity of the peptide.

In filter decanting, a mixture containing a peptide precipitate is provided to a vessel having a filter decanting apparatus. In the case where a precipitation step is performed, the precipitation can be performed in the vessel having the filter decanting apparatus or the precipitation can occur in a different vessel and mixture containing the precipitate can be transferred to vessel having the filter decanting apparatus.

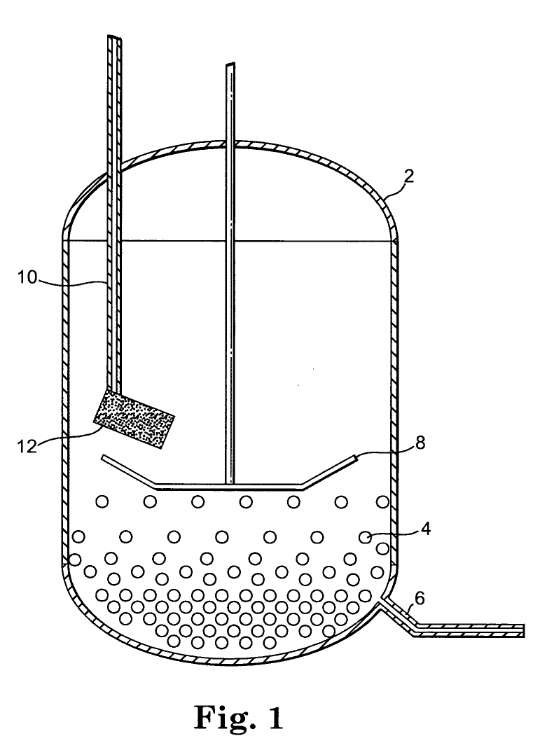

FIG. 1 illustrates a vessel 2 having a filter decanting apparatus. Generally, the vessel 2 contains a mixture that includes a precipitated peptide particle 4. In some cases, the mixture is formed by feeding a precipitating agent into the vessel 2, via a feed line 6. If a precipitating step is performed, an agitator 8 can be operated to blend the precipitating agent with the peptide. Parameters such as the temperature of the peptide in the vessel 2, the temperature of the precipitating agent, the feed rate of the precipitating agent, and the rate of the agitator 8 can be controlled either manually or automatically. Exemplary parameters are disclosed herein. An amount of precipitating agent can be added to the vessel 2 in order to precipitate the peptide to form a precipitated peptide particle 4. Additional precipitating agent can be added if desired.

Upon the addition of a precipitating amount of precipitating agent, the precipitated peptide 4 can settle by gravity in the vessel 2. The extent of settling can depend on a number of factors, including the peptide precipitate particle size and operational rate of the agitator 8. In some processes it may be desirable to allow the precipitate to settle to a certain extent before operating the filter decant apparatus.

When the precipitation step has proceeded to an acceptable point, the filter decanting process can be initiated. The filter decant method essentially removes most or all of the supernatant of the mixture from the vessel 2, leaving a peptide precipitate concentrate in the vessel. Filter decanting can be performed by extending tube 10 into the vessel 2, wherein the end of the tube 10 that is in contact with the supernatant has a decant filter 12. In order to remove the supernatant, a vacuum or pump attachment (not shown) can be applied to tube 10 to suction off the liquid mixture through decant filter 12. If desired, the tube 10 and decant filter 12 can be raised or lowered during the process in order to place the decant filter 12 in a desired portion of the mixture or a series of decant filters can be placed at fixed levels. For example, during the beginning of the filter decant process the decant filter 12 can be placed in the upper levels of the mixture, where there can be a lower concentration of precipitated peptide and then gradually lowered as more mixture is removed from the vessel. As more mixture is removed, the precipitated peptide particle 4 remains in the lower portion of the vessel 2, on the sides of the vessel, or both. The decant filter 12 blocks most, if not all, of the precipitated peptide particle 4 from being removed with the liquid mixture.

The dimensions of the decant filter 12 can be sized in relation to the vessel. It is understood that by increasing the area on the surface of the decant filter 12 the flow rate of removal of supernatant from the vessel 2 can be increased. The decant filter 12 also includes a membrane having pores of a size that prevent most, if not all, of the precipitated peptide particle 4 from flowing through the decant filter 12.

In preferred embodiments the vessel 2, feed line 6, tube 10, and filter 12 are of a size to accommodate a scaled-up synthesis.

Following this filter decant step, the precipitated peptide particle 4 remaining in the vessel 2 can be washed once, or more than once, with a suitable liquid. Suitable liquids, for example the precipitating agent, typically keep the peptide in a precipitated form and are able to remove a portion or all of the remaining impurities. A desired number of washes can be performed at a desired temperature, amount of wash liquid, agitation rate, and time period. After each wash a step of filter decanting can be performed.

After the washes are performed, the precipitated peptide can be processed as necessary. For example, the peptide can be subject to a further treatment, such as chemical modification, purification by chromatography, isolation, or storage.

The invention generally describes methods that have a filter decanting step. However, in another aspect, the methods described herein also provide a method for deprotecting a peptide that includes a filter decant step. The deprotection comprises the steps of contacting a peptide comprising side chain protecting groups with a deprotection composition that includes an acidolytic agent in an amount greater than 90/100 parts by weight, wherein said contacting forms a deprotected peptide; precipitating the deprotected peptide to form a mixture that includes a supernatant having the acidolytic agent and a peptide precipitate; and then filter decanting the supernatant to isolate the peptide precipitate.

The precipitation and filter decanting methods of the invention allows for the use of deprotection solutions that can have relatively high concentrations of the acidolytic reagent. In some cases, high concentrations of an acidolytic agent can be detrimental to the quality of the resulting deprotected peptide. However, the precipitation and filter decanting methods described herein can effectively remove high concentrations of the acidolytic reagents and deprotection reaction by-products. Use of relatively higher levels of acidolytic reagent in the deprotection reaction allows, for example, a reduction in the amount of time needed for the deprotection reaction to occur.

If the precipitation and filter decanting steps described herein are incorporated into a peptide synthesis having a deprotection step, the deprotection reaction can be optimized for efficiency. Accordingly, the invention provides optimized parameters for a solution-phase global deprotection reaction that provides efficient deprotection of the peptide. Specifically, high concentrations of an acidolytic agent are used to remove the side-chain protecting groups in a relatively short deprotection reaction time and subsequently the precipitation and decant filtration method effectively removes the deprotection reagents and by-products.

A deprotection step is performed to remove one or more protecting groups from the peptide. The protecting group can be any sort of group that can predominantly prevent the atom to which it is attached, typically oxygen or nitrogen, from participating in an undesired reaction or bonding upon further processing. Protecting groups include side chain protecting groups and terminal protecting groups, such as N-terminal and C-terminal protecting groups. Protecting groups can also predominantly prevent reaction or bonding of, for example, carboxylic acids, amines, alcohols, and thiols.

The deprotection reaction can be used to remove a side chain protecting group, which is typically a chemical moiety coupled to the side chain (R group) of an amino acid and predominantly prevents a portion of the side chain from reacting with chemicals used in steps of peptide synthesis procedures. Examples of side chain protecting groups include acetyl (Ac); benzoyl (Bz); tert-butyl; triphenylmethyl (trityl); tetrahydropyranyl; benzyl ether (Bzl); 2,6-dichlorobenzyl (DCB); t-butoxycarbonyl (BOC); nitro; p-toluenesulfonyl (Tos); adamantyloxycarbonyl; xanthyl (Xan); benzyl; methyl; ethyl; t-butyl ester; benzyloxycarbonyl (Z); 2-chlorobenzyloxycarbonyl (2-Cl-Z); Tos; t-amyloxycarbonyl (Aoc); aromatic or aliphatic urethan-type protecting groups; photolabile groups such as nitro veritryl oxycarbonyl (NVOC); and fluoride labile groups such as trimethylsilylethyl oxycarbonyl (TEOC).

Commonly used side chain protecting groups include t-Bu group for Tyr(Y), Thr(T), Ser(S) and Asp(D) amino acid residues; the trt group for His(H), Gln(Q) and Asn(N) amino acid residues; and the Boc group for Lys(K) and Trp(W) amino acid residues.

In some embodiments, the precipitation and filter decanting methods of the invention can be performed following a global deprotection reaction that removes the side chain protecting groups from a peptide. Global deprotection is typically performed at later stages in peptide synthesis wherein addition of peptide residues has been completed. Global deprotection can be performed, for example, after a peptide is cleaved from a resin in a solid-phase synthesis, or following a solution phase step, such as the coupling of peptide intermediate fragments.

The removal of side chain protecting groups by global deprotection typically utilizes a deprotection solution that includes an acidolytic agent to cleave the side chain protecting groups. Commonly used acidolytic reagents for global deprotection include triflouroacetic acid (TFA), HCl, lewis acids such as BF₃Et₂O or Me₃SiBr, liquid hydrofluoric acid (HF), hydrogen bromide (HBr), trifluoromethane sulfuric acid, and combinations thereof. The deprotection solution also includes one or more suitable chemical scavengers, for example, dithiothreitol, anisole, p-cresol, ethanedithiol, or dimethyl sulfide. The deprotection solution can also include water. As used herein, amounts of reagents present in the deprotection composition are typically expressed in a ratio, wherein the amount of an individual component is expressed as a numerator in “parts”, such as “parts weight” or “parts volume” and the denominator is the total parts in the composition. For example, a deprotection solution containing TFA:H₂O:DTT in a ratio of 90:5:5 (weight/weight/weight) has TFA at 90/100 parts by weight, H₂O at 5/100 parts by weight, and DTT at 5/100 parts by weight.

In some embodiments, the deprotection reaction can be performed wherein the amount of the acidolytic agent, preferably TFA, in the deprotection composition is greater than 90/100 parts by weight. More preferably, the acidolytic agent, preferably TFA, is present in an amount of 93/100 parts by weight or greater. Most preferably, the acidolytic agent, preferably TFA, is present in an amount in the range of 93/100 by weight to 95/100 parts by weight.

As a consequence of increasing the amount of acidolytic agent in the deprotection composition, the amount of water, scavenger, or both, would need to be reduced. According to the invention, the deprotection compositions having relatively high amounts of the acidolytic agent, and limited amounts of water were successfully used to deprotect the peptide and provide a product, following filter decanting, that had good purity. Therefore, in other embodiments, the amount of water in the deprotection composition can be less than 5/100 parts by weight. More preferably the water is in an amount of 3.5/100 parts by weight or less. Most preferably, the water is in an amount in the range of 3.5/100 parts by weight to 0.8/100 parts by weight.

In addition, the amount of scavenger, preferably DTT, in the deprotection composition can be greater than 5/100 parts by weight.

In another preferred embodiment the deprotection composition includes 94 parts by weight or greater acidolytic agent, 1.0 parts by weight or less water, and 5.0 parts by weight or greater scavenger. In preferred embodiments the acidolytic agent is TFA and the scavenger is DTT.

The higher amounts of the acidolytic component in the deprotection solution allow the deprotection reaction to be performed more rapidly. Global deprotection can be performed, for example, in a large-scale peptide synthesis, for a period in the range of about 5 to 10 hours, or 5 to less than 10 hours; preferably global deprotection is performed in the range of 5 to 6.5 hours. Temperatures for the global deprotection can be maintained in the range of 15 to 30° C.

In some embodiments, the precipitation and filter decanting method is preceded by a deprotection reaction wherein the deprotection solution is combined with a cosolvent. For example, the cosolvent can be combined with the deprotection solution, wherein the concentration of the peptide in the cosolvent is in the range of 3 L to 4 L of cosolvent per kg of peptide. In one embodiment the cosolvent includes, or is preferably, dichloromethane.

In some embodiments the peptide is deprotected, as described, and then provided to the precipitation and filter decanting steps as a peptide in a deprotection solution. In other embodiments of the invention, the deprotection step does not need to be performed before the precipitation and filter decanting steps; rather, the peptide can be provided in any solution which can be subject to the precipitation and filtration steps. These steps include contacting a peptide in a solution with a precipitating agent for the peptide to form a mixture which includes a supernatant and a peptide precipitate, and agitating the mixture; and then filter decanting the supernatant to isolate the peptide precipitate.

The peptide in solution is generally solubilized in that solution, and is, for example, preferably not coupled to a resin. A precipitating agent for the peptide is then fed into the solution containing the peptide and agitation is applied to adequately blend the mixture which contains the precipitating agent, the solution, and the peptide. The peptide precipitates from the mixture after a precipitating amount of the precipitating agent has been fed into the solution. Additional precipitating agent can continue to be added to the mixture. The precipitated peptide has a particle size that allows it to settle in the mixture by gravity. The liquid mixture that does not contain the precipitated peptide particles is then removed by filter decanting.

The precipitating agent for the peptide is added in an amount (“a precipitating amount”) sufficient to precipitate the peptide. One example of a precipitating agent is methyl-tert-butyl ether (MTBE). In another aspect, the volume of precipitating agent added to the solution is based on the amount of peptide in solution. Therefore in corresponding embodiments, the precipitating solvent is added in an amount to provide a range of 50 L to 70 L of slurry per kg of peptide (crude isolation). The amount of precipitating agent in the final slurry is typically in the range of 57% to 70%.

In addition, it has been discovered that in the precipitation and filter decanting steps, certain parameters are particularly useful for allowing these steps to proceed efficiently. These parameters include temperatures at which the precipitating solvent is combined with the peptide solution, rates at which the precipitating solvent is fed into the peptide solution, agitation rates, and temperatures during the precipitation and agitation step. Exemplary precipitation and filter decanting utilizes MTBE which is combined with peptide in a deprotection solution that includes TFA and a deprotected peptide.

It is advantageous that when the solvent is initially added to the peptide solution, both are chilled. Preferably, both the precipitating agent and the peptide solution are at a temperature in the range of −5° C. to 5° C. when combined. In particular, temperatures of near −5° C. are preferred. After the cold solutions are combined, the mixture is gradually warmed. It is thought that these parameters provides a mixture that has particularly good filter decanting properties and peptides precipitates with improved quality, for example, higher purity. It is thought that these advantages are achieved, at least in part, by minimizing peptide degradation and unwanted modification during early stages of the precipitation process, and in some aspects, during extended times that are often encountered in scale-up processing, such as large scale processing.

According to the invention, it has also been found that adding the precipitating agent to the peptide solution at a rate in the range of 0.3 kg/min to 2.5 kg/min, and in some preferred embodiments about 2 kg/min, provides a mixture that has particularly good filter decanting properties and also improves the quality of the peptide.

At the time the precipitating solvent is added to the solution containing the peptide, or afterwards, the mixture is preferably warmed to a temperature of 12° C. or greater. More preferably, the temperature is in the range of 12° C. to 20° C., and even more preferably in the range of 15° C. to 20° C. It is desirable to warm the solution to 12° C. or greater before precipitation of the peptide begins to occur. In a preferred aspect, the mixture can be warmed at a rate in the range of 7° C. to 15° C. per hour. The temperature of the mixture can be maintained in the desired range for at least a portion of this step, and preferably until the filter decant step is performed.

After the precipitating agent has been blended and the peptide precipitate formed and aged for a suitable amount of time, the filter decanting step can be performed. Details of the filter decanting step are described herein. It has been discovered that the parameters described for the peptide precipitation provide precipitated peptide particles that allow rapid filter decanting of the liquid mixture (supernatant). In an exemplary embodiment, the filter decanting process utilizes a decant filter having a pore size in the range of 10-20 μm, which can allow the supernatant to be efficiently removed from the vessel.

After the liquid mixture (supernatant) has been decanted, the cake of precipitated peptide particles can be washed with the same or a different precipitating solvent. The wash can be performed at a temperature in the range of, for example, −10° C. to 20° C., and preferably 0° C. or less. The filter decanting can be repeated to remove the wash solvents.

As an optional step after washing, the cake of precipitated peptide particles can be dried or partially dried. In some embodiments, the precipitated peptide particles are partially dried and provided to a subsequent reaction as a wet cake. In these embodiments, the precipitated peptide can be provided to a subsequent reaction having a LOD (loss on drying) in the range of 2% to 65%. Preferred drying temperatures are in the range of −5° C. to 45° C., and more preferably in the range of −5° C to −3° C. Drying can be performed for a relatively short period, for example, up to one hour.

In some embodiments, the peptide that has been subject to the precipitation and filter decanting steps as described herein can be subject to a further chemical reactions or modifications. One exemplary modification is performing a decarboxylation reaction on the peptide. For example, in embodiments wherein the peptide is subject to a global deprotection reaction a peptide may still retain a carbamate functionality on an amine side chain. After the peptide carbamate is taken through the precipitation and filter decanting methods as described herein, it can be subject to a decarboxylation step.

A decarboxylation step can be performed on the peptide using a decarboxylation reagent, such as acetic acid. Following the decarboxylation step, the peptide can be subject to a purification step, for example, using the decant filtration methods as described herein.

Additional procedures involved in the solid phase, solution phase, and/or hybrid synthesis of peptides are discussed in the following U.S. provisional applications: (1) U.S. provisional application No. 60/533,655, filed Dec. 31, 2003, titled “Methods For Recovering Cleaved Peptide From A Support After Solid Phase Synthesis” bearing attorney docket no. RCC008/P1, in the names of inventors including Robert Orr Cain; (2) U.S. provisional application No. 60/533,653, filed Dec. 31, 2003, titled “Process and Systems for Recovery of Peptides” bearing attorney docket no. RCC009/P1, in the names of inventors including Hiralal Khatri; (3) U.S. provisional application No. 60/533,654, filed Dec. 31, 2003, titled “Process and Systems for Peptide Synthesis” bearing attorney docket no. RCC0001/P1, in the names of inventors including Mark A. Schwindt; and (4) U.S. provisional application No. 60/533,710, filed Dec. 31, 2003, titled “Peptide Synthesis and Deprotection Using a Cosolvent” bearing attorney docket no. RCC0012/P1, in the names of inventors including Mark A. Schwindt.

The following non-limiting examples are provided to illustrate aspects of the present invention.

### EXAMPLE 1

### Preparation of Enfuvirtide

This example describes the formation and global deprotection of protected enfuvirtide with an acidolytic deprotection composition to form an enfuvirtide carbamate salt. This example also describes additional processing steps, including filter decanting and decarboxylation of the enfuvirtide carbamate. Four batches (A-D) of the peptide were prepared.

As starting material, a side-chain protected, N-terminal acetylated enfuvirtide peptide (Ac-AA(1-36)NH₂), having the formula: Ac-Tyr(tBu)-Thr(tBu)-Ser(tBu)-Leu-Ile-His(trt)-Ser(tBu)-Leu-Ile-Glu(OtBu)-Glu(OtBu)-Ser(tBu)-Gln(trt)-Asn(trt)-Gln(trt)- Gln-Glu(OtBu)-Lys(Boc)-Asn(trt)-Glu(OtBu)-Gln(trt)-Glu(OtBu)-Leu-Leu-Glu(OtBu)- Leu-Asp(tBu)-Lys(Boc)-Trp(Boc)-Ala-Ser(tBu)-Leu-Trp(Boc)-Asn(trt)-Trp(Boc)-Phe-NH₂ (SEQ ID NO:1 with side chain protecting groups) was prepared using a combination of solid-phase and solution-phase peptide synthesis steps. Ac-AA(1-36)NH₂ can be prepared according to the methods described in U.S. Pat. No. 6,015,881.

Briefly, enfuvirtide peptide intermediate fragments H-AA(17-36)-NH₂ (protected) was coupled to Ac-AA(1-16)OH (protected) in a solution phase reaction to form Ac-AA(1-36)NH₂ (protected). These solid phase synthesis and solution phase coupling steps to form the H-AA(17-36)-NH₂ (protected) and Ac-AA(1-16)OH (protected) intermediate fragments can be performed according to the methods described in U.S. Pat. No. 6,015,881.

For each batch (batches A-D) the following procedure was carried out. The solid raw materials (Ac-AA(1-16)OH (protected); H-AA(17-36)-NH₂ (protected); and 6-chloro-1-hydroxybenzotriazole (6-Cl—HOBT)) were charged to a vessel (see Table 1). The solids were dissolved in dimethylformamide (DMF) and the resulting solution was pre-cooled to 0° C. Diisopropylethylamine (DIEA) was added, followed by O-benzotriazol-1-yl-N,N,N′,N′-tetramethyluronium hexafluorophosphate (HBTU) as a solid. The reaction mixture was stirred at 0° C. then warmed to 20° C. over about 2 h (see Table 2; for the HPLC chromatogram, % AN (area norm) refers to the area percent under a peak at a particular wavelength). The reaction mixture was sampled and tested for the coupling reaction completion to the Ac-AA(1-36)NH₂ (protected) product. When complete, water was charged to the coupling mixture to precipitate Ac-AA(1-36)NH₂ (protected) as a solid (see Table 3). The solid product was dissolved and extracted into methylene chloride (see Table 4). The DMF/water layer was separated and removed. The methylene chloride solution of Ac-AA(1-36)NH₂ (protected) was washed with water several times to remove residual DMF. The Ac-AA(1-36)NH₂ (protected) solution was concentrated by vacuum distillation to remove most of the methylene chloride (see Table 5). The target volume of remaining methylene chloride was 3-4 L/kg of peptide. The jacket temperature was kept below 45° C. and the vessel temperature was kept below 40° C. during the distillation. The concentrated methylene chloride solution of Ac-AA(1-36)NH₂ (protected) was added to a mixture of trifluoroacetic acid (TFA), dithiothreitol and water (in a 94.0:5.2:0.8 w/w/w ratio) (see Table 6). The de-protection reaction mixture was stirred at 25° C. (±3) for 4-6 h, and cooled to 0° C. (±5). MTBE was charged to quench the reaction and to precipitate Ac-AA(1-36)NH₂ carbamate (see Table 7). The solids were filtered, washed with MTBE, and partially dried up to 60% LOD. In all batches, a Hasteloy 10-20 μm filter was used to isolate Ac-AA(1-36)NH₂ carbamate by a filter decanting process (apparatus and method in accordance with FIG. 1; see also Table 8). The wet cake was washed with MTBE three times.

Isopropanol, DIEA, and acetic acid were charged to the semi dried solid intermediate in the decant vessel to obtain a pH typically between 4 and 5 (see Table 10). The slurry mixture was warmed to 35° C. (±5), sampled, and tested for de-carboxylation reaction completion. The slurry mixture was cooled to 10° C. (±5), the solid Ac-AA(1-36)NH₂ product filtered, and washed with isopropanol. In all batches, a Heinkel filter with polypropylene filter cloth, 5-7 μm size, was used to isolate the Ac-AA(1-36)NH₂ product (see Table 11). Isolation involved filtering to remove the liquors, followed by washing with isopropanol. The cake was then blown down to remove as much isopropanol as possible from the cake. The Ac-AA(1-36)NH₂ product was dried under vacuum and packaged (see Table 12). DV-111 rotary dryer was used as the dryer for preparation of batches A-D. The wet cake from Heinkel isolation was dried under vacuum at less than 40° C. until the LOD was less than 2.0%. Data for purity and yield is provided in Table 13.

TABLE 1
Raw Material Loading Data

| | Batch | | | |
|---|---|---|---|---|
| | A | B | C | D |
| Ac-AA(1-16)OH charge, (kg) | 9.0 | 8.6 | 8.6 | 8.4 |
| Ac-AA(1-16)OH, (mol) | 2.73 | 2.61 | 2.61 | 2.55 |
| H-AA(17-36)NH₂ charge, (kg) | 11.4 | 11.4 | 11.4 | 11.4 |
| H-AA(17-36)NH₂, (mol) | 2.75 | 2.75 | 2.75 | 2.75 |
| 6-Chloro-HOBt charge, (kg) | 0.8 | 0.7 | 0.7 | 0.7 |
| DIEA charge, (kg) | 0.65 | 0.65 | 0.65 | 0.65 |
| HBTU charge, (kg) | 1.2 | 1.2 | 1.2 | 1.2 |
| DMF charge, (kg) | 114.0 | 114.0 | 113.9 | 113.1 |

TABLE 2
Coupling Reaction Data

| | Batch | | | |
|---|---|---|---|---|
| | A | B | C | D |
| Stir time, (h:min) | 10:15 | 2:0 | 1:11 | 1:15 |
| Warm up time to 25° C., | N/A | 0:40 | 0:23 | 0:33 |
| (h:min) | | | | |
| Age time before quench, | N/A | 2:0 | 2:0 | 2:0 |
| (h:min.) | | | | |

Coupling Reaction Completion to Ac-AA(1-36)NH₂ HPLC Results,
(% AN)

| | | | | |
|---|---|---|---|---|
| Ac-AA(1-36)NH₂ | 68.9 | 57.6 | 69.8 | 68.8 |
| Ac-AA(1-16)OH | 2.7 | 0.76 | 0.5 | 1.6 |
| H-AA(17-36)NH₂ | 0.1 | 0.11 | 0.9 | 0.6 |

TABLE 3
Water Quench Data

| | Batch | | | |
|---|---|---|---|---|
| | A | B | C | D |
| Water charge (L) | 120 | 120 | 120 | 120 |
| Water addition time (min) | 31 | 31 | 50 | 30 |
| Exothermic range (° C.) | 5-17 | 18-25 | 16-22 | 20-21 |

TABLE 4
Extraction Data

| | Batch | | | |
|---|---|---|---|---|
| | A | B | C | D |
| Methylene chloride charge (kg) | 425.0 | 426.0 | 425.0 | 429.0 |
| Stir period (min) | 15 | 15 | 15 | 30 |
| Stir temperature (° C.) | 14 | 14-15 | 15-16 | 12-13 |
| Settle period (min) | 47 | 130 | 125 | 120 |
| Settle temperature (° C.) | 14-15 | 14-15 | 16-18 | 13-14 |
| Water charge #1 (L) | 120.0 | 120.0 | 158.0 | 158.0 |
| Stir period (min) | 15 | 15 | 15 | 15 |
| Stir temperature (° C.) | 16-17 | 19 | 19 | 16-17 |
| Settle period (min) | 45 | 83 | 45 | 55 |
| Settle temperature (° C.) | 17 | 18-19 | 19 | 17-18 |
| Water charge #2 (L) | 120.0 | 120.0 | 158.0 | 158.0 |
| Stir period (min) | 16 | 15 | 17 | 15 |
| Stir temperature (° C.) | 16 | 16 | 17 | 16 |
| Settle period (min) | 138 | 45 | 105 | 360* |
| Settle temperature (° C.) | 16 | 16 | 17-18 | 16-18 |
| Water charge #3 (L) | 120.0 | 120.0 | 158.0 | 158.0 |
| Stir period (min) | 15 | 16 | 16 | 15 |
| Stir temperature (° C.) | 16 | 18 | 17-18 | 17-18 |
| Settle period (min) | 61 | 49 | 49 | 45 |
| Settle temperature (° C.) | 16 | 18-19 | 17-18 | 17-18 |
| Water charge #4 (L) | 120.0 | 120.0 | 158.0 | 158.0 |
| Stir period (min) | 15 | 15 | 15 | 81 |
| Stir temperature (° C.) | 15 | 17 | 16 | 18-23 |
| Settle period (min) | 45 | 47 | 45 | 320 |
| Settle temperature (° C.) | 15 | 17-18 | 16-17 | 23-24 |
| Water charge #5 (L) | 120.0 | 120.0 | 158.0 | 158.0 |
| Stir period (min) | 15 | 15 | 15 | 15 |
| Stir temperature (° C.) | 15 | 17-19 | 16-17 | 20-23 |
| Settle period (min) | 46 | 70 | 120 | 324 |
| Settle temperature (° C.) | 15-16 | 17-19 | 16-18 | 20-24 |
| Gas Chrom. % w/w DMF | 2.5 | 1.8 | 1.1 | 1.5 |
| DCM volume, L | 300 | 340 | 320 | 400 |

Batch D had an emulsion problem during the 4^{th} water wash. This emulsion was due to a slight increase in agitation speed. The problem was solved by extending settle time, increasing the batch temperature and by taking emulsified layer with organic layer and adding more methylene chloride to separate the layers. Excess methylene chloride was removed by distillation.

TABLE 5
Distillation Data

| | Batch | | | |
|---|---|---|---|---|
| | A | B | C | D |
| Distillation time, h:min | 20:15* | 15:15* | 03:38 | 05:18 |
| Distillation temperature, (° C.) | 6-23 | 6-19 | 4-18 | 11-24 |
| Vacuum, (mmHg) | 150 | 150 | 170 | 230 |
| Batch Volume, (L) | 70 | 70 | 70 | 70 |

Distillate Sample Results

| | | | | |
|---|---|---|---|---|
| Ac-AA(1-36)NH₂, (g/L) | 4.1 | 1.8/0.47 | 5.3 | 0.1 |
| Distillate volume, (L) | 230 | 240/260 | 250 | 260 |

Distillation Completion Sample Results

| | | | | |
|---|---|---|---|---|
| Water, (%) | 1.13 | 0.59 | 0.08 | 0.05 |
| DMF, (wt./wt. %) | 10.2 | 5.6 | 3.7 | 5.6 |
| DCM volume, L | 70 | 70 | 70 | 70 |
| DCM charge, kg | 350 | 350 | N/A | N/A |
| GC % w/w Results for DMF | 4.4 | 6.2 | | |
| KF, % water | 0.37 | 0.022 | | |
| DCM volume, L | 70 | 70 | | |

*DCM addition and extraction repeated due to higher water and/or DMF levels after first distillation.

TABLE 6
De-protection Reaction Data

| | Batch | | | |
|---|---|---|---|---|
| | A | B | C | D |
| Trifluoroacetic acid charge (kg) | 189.1 | 244.8 | 244.9 | 247.3 |
| Dithiothreitol charge (kg) | 10.2 | 13.6 | 13.4 | 13.6 |
| Potable water charge (kg) | 4.0 | 2.0 | 2.0 | 2.0 |
| DCM rinse (kg) | 37.0 | 37.0 | 37.0 | 32.0 |
| Reaction time (h) | 10 | 6 | 5 | 5 |
| Stir temperature (° C.) | 16-23 | 26 | 25 | 22 |

Rxn completion, Ac-AA(1-36)NH₂, carbamate

| | | | | |
|---|---|---|---|---|
| HPLC % AN Results for | 61.5 | 63.1 | 72.3 | 70.3 |
| Ac-AA(1-36)NH₂ | | | | |
| % AN for 9.5 (RRT 1.23) | 2.7 | 1.5 | 0.6 | 1.7 |
| peak | | | | |

TABLE 7
MTBE Quench Data

| | Batch | | | |
|---|---|---|---|---|
| | A | B | C | D |
| MTBE Charge (kg) | 318.8 | 318.4 | 319.0 | 318.0 |
| MTBE Temperature | 0 (±3) | 0 (±3) | 0 (±3) | 0 (±3) |
| (° C.) | | | | |
| Batch temperature | −4 | 1 | −5 | −5 |
| prior to quench | | | | |
| (° C.) | | | | |
| Transfer rate | 0.3-1.7 | 2.0-2.5 | 2.0 | 1.8-2.0 |
| (kg/min) | | | | |
| Jacket heat up ramp to | 7 | 15 | 15 | 15 |
| 15° C., ° C./hr | | | | |
| Total Feed time (h:min) | 4:37 | 2:28 | 3:0 | 3:0 |
| Age time (min) | 30 | 30 | 30 | 35 |

MTBE was added through a mass flow meter and the bath was ramped during the feed/precipitation.

TABLE 8
Decant Filtration Data

| | Batch | | | |
|---|---|---|---|---|
| | A | B | C | D |
| Initial Filtration time, | 7:00 | 5:16 | 3:45 | 3:00 |
| (h:min) | | | | |
| 1^{st} MTBE charge for | 60.5 | 59.7 | 60.6 | 60.5 |
| washing (kg) | | | | |
| 2nd MTBE wash charge | 60.5 | 57.0 | 60.8 | 60.5 |
| (kg) | | | | |
| 3rd MTBE wash charge | 60.4 | 63.6 | 60.5 | 57.3 |
| (kg) | | | | |
| Total decant wash time | 6:10 | 9:30 | 5:24 | 8:50 |
| h:min | | | | |

TABLE 9
Drying Data

| | Batch | | | |
|---|---|---|---|---|
| | A | B | C | D |
| Jacket temperature (° C.) | −5 | −5 | −3 | −3 |
| Drying time (h:min) | 0:45 | 0:50 | 0:33 | 0:30 |
| Final LOD (%) | 47.63 | 60.23 | 57.24 | 56.98 |

TABLE 10
De-carboxylation Reaction Data

| | Batch | | | |
|---|---|---|---|---|
| | A | B | C | D |
| Isopropanol charge (kg) | 145.2 | 218.0 | 220.3 | 221.8 |
| DIEA charge (kg) | 5.3 | 8.0 | 7.9 | 8.0 |
| Acetic acid charge (kg) | 11.0 | 11.0 | 11.1 | 11.0 |
| Batch pH | 4.8 | 4.5 | 4.99 | 5.08 |

Rxn Time, h:min @ Temperature ° C.

| | | | | |
|---|---|---|---|---|
| Warm up | 6:02 @ 0-35 | 1:10 @ 14-30 | 1:0 @ 14-30 | 0:30 @ 19-33 |
| Age | * | 1:10 @ 32 | 1:05 @ 30-31 | 0:50 @ 33-34 |

*Not recorded

TABLE 11
Heinkel Filtration Data

| | Batch | | | |
|---|---|---|---|---|
| | A | B | C | D |
| Isopropanol charge (kg) | 200.1 | 203.6 | 77.9 | |
| Total # of spins | 52 | 53 | 64 | 46 |
| Wet cake (kg) | 28.6 | 27.5 | 29.8 | 26.9 |
| Filtration time (h:min) | 10:00 | 10:28 | 16:00 | 07:50 |
| Product loss in mother | 0.04 | 0.03 | 0.02- | 0.06 |
| liquors (g/L) | | | | |
| Volume L | 635 | 400 | 600 | 430 |

TABLE 12
Drying Data

| | Batch | | | |
|---|---|---|---|---|
| | A | B | C | D |
| Average jacket | 38 | 38 | 38 | 38 |
| temperature (° C.) | | | | |
| Vacuum (inch Hg) | 9-22 | 6-20 | 8-22 | 8-30 |
| LOD (%) | 0.32 | 0.68 | 0.56 | 0.75 |
| Drying time (d:h:min) | 04:06:25 | 02:16:11 | 03:07:05 | 02:15:03 |

TABLE 13
Yields and Purity Data

| | Batch | | | |
|---|---|---|---|---|
| | A | B | C | D |
| Yield Ac-AA(1-36)NH₂ (%) | 92.2 | 94.7 | 105.2 | 99.5 |
| Quantity of Ac-AA(1- | 11.4 | 11.7 | 13.0 | 12.3 |
| 36)NH₂ (kg) | | | | |
| Purity (% AN) | 58.1 | 61.1 | 70.6 | 68.5 |
| Purity (% w/w) | 44.8 | 43.8 | 50.8 | 51.3 |
| Contained Yield, | 5.11 | 5.13 | 6.60 | 6.31 |
| (y × wt assay), kg | | | | |

1. A method for isolating a peptide comprising steps of:
(a) providing a mixture comprising a supernatant and a peptide precipitate; and
(b) filter decanting the supernatant to isolate the peptide precipitate.

2. The method of claim 1 wherein the mixture is formed by contacting a peptide in a solution with a precipitating agent for the peptide, and wherein the mixture is agitated.

3. The method of claim 2 wherein the precipitating agent is present in the mixture in an amount in the range of 57% to 70%.

4. The method of claim 2 where, in the step of contacting, the peptide in solution and the precipitating agent are at a temperature in the range of −5° C. to 5° C.

5. The method of claim 2 where, in the step of contacting, the peptide in solution and the precipitating agent are at a temperature in the range of −5° C. to −3° C.

6. The method of claim 1 comprising a step of warming the mixture to a temperature of 12° C. or greater.

7. The method of claim 6 wherein the mixture is warmed to a temperature in the range of 12° C. to 20° C.

8. The method of claim 7 wherein the mixture is warmed to a temperature in the range of 15° C. to 20° C.

9. The method of claim 6, wherein the mixture is warmed to a temperature of 12° C. or greater before an amount of precipitating agent of one half an amount of peptide in solution, contacts the peptide in solution.

10. The method of claim 2 wherein the step of contacting comprises feeding the precipitating agent into the solution at a rate in the range of 0.3 kg/min to 2.5 kg/min.

11. The method of claim 2 wherein the precipitating agent is MTBE.

12. The method of claim 1 wherein the step of filter decanting comprises removing the supernatant utilizing a decant filter having a pore size in the range of 10-20 μm.

13. The method of claim 1 comprising a step of deprotecting the peptide, wherein the step of deprotecting provides the peptide in solution.

14. The method of claim 13 wherein deprotecting the peptide is performed with a deprotection composition comprising an acidolytic compound, water, and a scavenger.

15. The method of claim 14 wherein the acidolytic compound comprises TFA and wherein the TFA is present in the deprotection composition at greater than 90/100 parts by weight.

16. The method of claim 15 wherein the TFA is present in the deprotection composition at 93/100 parts by weight or greater.

17. The method of claim 16 wherein the TFA is present in the deprotection composition at 95/100 parts by weight or greater.

18. The method of claim 17 wherein the TFA is present in the deprotection composition in the range of 93/100 parts by weight to 95/100 parts by weight.

19. The method of claim 14 wherein the water is present in the deprotection composition at less than 5/100 parts by weight.

20. The method of claim 19 wherein the water is present in the deprotection composition at 3.5/100 parts by weight or less.

21. The method of claim 20 wherein the water is present in the deprotection composition in the range of 3.5/100 parts by weight to 0.8/100 parts by weight.

22. The method of claim 14 wherein the scavenger is DTT and wherein DTT is present in the deprotection composition at less than 5/100 parts by weight.

23. The method of claim 14 wherein the deprotection composition is combined with a cosolvent.

24. The method of claim 23 wherein the cosolvent is dichloromethane.

25. The method of claim 1 comprising a step of washing the peptide precipitate with the precipitating agent or a different precipitating agent.

26. The method of claim 1 comprising a step of partially drying the peptide precipitate.

27. The method of claim 1 wherein the peptide precipitate comprises a carbamate.

28. The method of claim 1 comprising a step of decarboxylating the peptide precipitate.

29. A peptide prepared using the steps of claim 1.

30. The method of claim 1 wherein the peptide comprises SEQ ID NO 1.

31. A method for deprotecting a peptide comprising the steps of:
(a) contacting a peptide comprising side chain protecting groups with a deprotection composition comprising an acidolytic agent in an amount greater than 90/100 parts by weight, wherein said contacting forms a deprotected peptide;
(b) precipitating the deprotected peptide to form a mixture comprising a supernatant having the acidolytic agent and a peptide precipitate; and
(c) filter decanting the supernatant to isolate the peptide precipitate.

32. A method for coupling peptide intermediate fragments comprising the steps of:
(a) coupling two or more peptide intermediates in solution to form a peptide product, wherein the peptide product comprises protecting groups;
(b) contacting the peptide product with a deprotection composition to form a deprotected peptide;
(c) contacting the deprotected peptide in the deprotecting composition with a precipitating agent for the peptide to form a mixture comprising a supernatant and a peptide precipitate, wherein the mixture is agitated; and
(d) filter decanting the supernatant to isolate the deprotected peptide precipitate.
